(19) United States
(12) Patent Application Publication
(10) Pub. No.: US 2022/0387744 A1
(43) Pub. Date: Dec. 8, 2022

(54) GAS CONDUIT FOR A RESPIRATORY SUPPORT DEVICE

(71) Applicant: MUNCH-NYGAARD, Bergen (NO)

(72) Inventor: Andreas MUNCH-NYGAARD, Bergen (NO)

(21) Appl. No.: 17/774,453

(22) PCT Filed: Jul. 24, 2020
(86) PCT No.: PCT/EP2020/070949

§ 371 Date: May 4, 2022

(30) Foreign Application Priority Data
Nov. 5, 2019 (NO) 20191317

(51) Int. Cl. A61M 16/08; A61M 16/06; A61M 16/20; A61M 16/00

(52) U.S. Cl. CPC A61M 16/0875; A61M 16/06; A61M 16/201; A61M 16/0078

(57) ABSTRACT

The present invention relates to a gas conduit for a respiratory support device comprising a face mask and a gas reservoir bag, said gas conduit comprising a proximal portion for fluid connection to the mask and a distal portion extending into the reservoir bag, wherein at least a side wall of said distal portion comprises one or more apertures. The present invention also relates to a respiratory support device comprising said conduit.

The present invention relates to a conduit for improved fluid communication between two components. More particularly, the present invention relates to a gas conduit for a medical device, such as a respiratory support device.

Respiratory support devices, such as oxygen masks are widely used for emergency medical treatments, inside and outside hospitals. High concentrations of oxygen are required to treat critically ill patients, and it is crucial that accurate and sufficient amounts of oxygen are delivered to the patient.

Conventional oxygen masks without a reservoir delivers oxygen with a flow rate of around 6 to 8 l/min oxygen, with a maximum of 50% actual oxygen administration. Conventional oxygen masks with a reservoir, sometimes known as non-rebreathing masks, the flow rate is increased to 8 to 12 l/min, and a maximum of 70% actual oxygen administration to the patient. The loss of efficiency is principally due to the configuration of existing oxygen masks, which results in gas leakage and/or impediment of gas flow through the device.

By way of example, a typical non-rebreathing mask comprises a face mask and an oxygen reservoir bag. The oxygen flows through a rigid cylinder in fluid communication between the mask and the reservoir. However, the opening of the cylinder in the reservoir can become partially or completely obstructed by the patient's own anatomy, clothing or cover, or even by the reservoir bag itself, thereby impeding oxygen flow. Worse yet, if reservoir is elevated relative to the mask (for example due to the patient's own anatomy, clothing or cover), then the mask is lifted off the patient's face, thereby resulting in gas leakage. This loss of performance renders nullifies the benefit of including a costly reservoir bag, and the efficiency of a leaking non-rebreather mask will be similar to that of a reservoirless mask.

Variations have been explored, in which the rigid cylinder is hingedly connected to the mask. However, such cylinders generally have a complex and expensive structure, and are often difficult to manipulate and position. Attempts have been made, in which the rigid cylinder is replaced with a flexible accordion-type tube. Again, such cylinder are difficult to manipulate and position, and in known designs, the accordion-tube can twist or kink and itself impede gas flow. Neither proposition satisfactorily solves the obstruction issue, because the bag itself can still fold over and hinder the tube opening. Furthermore, the flow of gas into and out of the reservoir is limited to the available size of the opening of the cylinder.

These problems have been brought to light and acutely emphasized by the global coronavirus (SARS-CoV-2) pandemic, which causes millions of patients to require respiratory support. It is crucial that patients receive the prescribed amounts of oxygen. Ensuring a minimum of 70% oxygen delivery would reduce the number of patients requiring longer, more extensive and/or intensive treatment, and decrease the number of patients being placed on a ventilator. In addition, medical facilities have seen their oxygen supplies deplete at such a rate that emergency refills of oxygen have to be delivered on a regular basis. Even a slight improvement in the oxygen delivery efficiency of a mask would alleviate the considerable and unnecessary accumulated losses of oxygen due to inefficient and/or ill-fitted masks.

It is an object of this invention to mitigate problems such as those described above and to provide an improved alternative to existing products. In particular, it is an object of the invention to provide a respiratory support device with improved oxygen delivery efficiency.

According to a first aspect of the invention, there is provided a gas conduit for a respiratory support device comprising a face mask and a gas reservoir bag, said gas conduit comprising a proximal portion for fluid connection to the mask and a distal portion extending into the reservoir bag, wherein at least a side wall of said distal portion comprises one or more apertures.

The provision of apertures in the side wall of the conduit increases the surface area available for the oxygen flow to enter or exit the conduit, and therefore an increased oxygen delivery. In particular, optimum oxygen flow is ensured through the apertures, even if the distal opening of the conduit is partially or completely obstructed by the patient's anatomy, clothes or cover. This is particularly relevant where the reservoir bag itself folds over and hinders the distal opening of the conduit.

Within the context of the invention, “proximal portion” refers to the portion of the conduit closer to the mask, and “distal portion” refers to the portion of the conduit further from the mask. “Proximal end” refers to the end of the conduit closest to the mask, and “distal end” refers to the portion of the conduit furthest from the mask.

The proximal portion of the gas conduit is preferably made of a flexible material. Alternatively, the conduit comprises one or more bends or one or more bendable portions.

If the conduit were a rigid straight tube extending from the mask, then any height difference between the mouth and the chest area of the patient would lift, or otherwise dislodge, the mask, and oxygen would leak from the gap between the patient's face and the mask. By contrast, a flexible conduit would bend or could be bent to steer the distal opening away from the obstruction.

Within the context of the present invention, “flexible” means flexible enough to enable bending, but rigid enough to provide structural rigidity sufficient so that the inner dimensions of the conduit remain substantially unchanged (thereby enabling optimum fluid passage through the conduit) and/or so that the conduit retains its given shape.

Additionally or alternatively, the conduit may comprise one or more pre-formed bends or be bendable.

The gas conduit preferably comprises or consists of a shapable material or structure. The conduit is preferably made, completely or partially, of a shapable material or structure. For example, the conduit can be bent manually by the health professional or user to form an angle α as required, and retain said angle. More generally, the conduit can be shaped manually by the health professional or user, and retain said shape.

The respiratory support device may be fitted to the patient, and the health professional will assess whether the oxygen flow is impeded in any way or if the mask is dislodged due to the height difference between the chest area and the mask and/or any other obstacles. If this is the case, the conduit may be shaped manually by the health professional to counteract said impediment, and will retain said shape.

In a preferred embodiment, the gas conduit comprises a frontal side and a dorsal side, and the apertures are formed at least on the dorsal side.

Within the context of the invention, “frontal side” refers to the side of the respiratory support device facing away from the patient, when the device is worn by the patient; and “dorsal side” refers to the side of device facing the patient.

This specific configuration, in which the apertures are formed on the dorsal side of the distal portion of the conduit, improves the bending ability of the conduit.

Additionally or alternatively, the apertures may be formed on the frontal side, which is less likely to be encumbered by obstacles that potentially could obstruct the flow passage.

In a preferred embodiment, the apertures are through-slits (i.e. elongated apertures). In a most preferred embodiment, the through-slits are formed on the dorsal side of the conduit. This configuration is particularly advantageous in that the conduit may be bend from a first configuration in which the apertures are substantially closed and the conduit is overall substantially straight, to a second configuration in which the apertures are open and the conduit is bent. The first configuration may be used when the distal opening is unobstructed. If the distal opening meets an obstacle, then the conduit may be bent, so that the apertures are opened and the distal opening is unobstructed. In the second configuration, oxygen passage is optimised both through the distal opening and the dorsal apertures. The apertures further facilitate the bending of the conduit.

Preferably, the distal portion of the gas conduit comprises a plurality of ribs. For example, two apertures may be separated by a rib, which provides structural rigidity to the distal portion of the conduit. Additionally or alternatively, the distal portion of the gas conduit may comprise a plurality of rings.

Preferably, the distal portion of the gas conduit comprises a mesh. In an embodiment, the conduit is made of a mesh. More preferably, the mesh is flexible and shapable. The flexibility and shapability enables the conduit to be shaped to avoid obstacles. The mesh structure provides optimum, unobstructed, oxygen flow.

Preferably, the gas conduit comprises a distal opening formed on the frontal side of the conduit. In other words, the gas distal opening may be positioned at the distal end of the conduit (as in conventional conduits), or on the frontal side of the conduit, in the side wall. This configuration minimises the risk of obstruction of the distal opening of the conduit.

Preferably, the gas conduit comprises a rounded distal end. This configuration is particularly advantageous when the distal opening is not formed at the distal end of the conduit, but for example on the frontal side of the conduit or when the conduit does not comprise a distal opening. This configuration enables the distal portion of the conduit to be naturally lifted when abutting an obstacle.

In a preferred embodiment, the distal and proximal portions of the gas conduit are integrally formed. This feature provides numerous advantages, including ease of manufacture, decreased manufacturing cost, fewer parts and therefore increased patient safety.

Preferably, the gas conduit comprises means for securing the proximal portion of the gas conduit to an element of the mask. The element of the mask may be formed on the mask, for example, it may be a neck of the mask. The element may be secured to the mask, for example a connector or a second component of the respiratory support device, for example a bag valve.

Preferably, the gas conduit comprises a valve to regulate the gas flow between the mask and the reservoir bag. For example, the valve may be a one-way valve. The one-way valve may be arranged and configured to allow passage of the gas (e.g. oxygen during inspiration) from the reservoir to the mask, and prevent passage of exhaled gas from the mask to the reservoir.

Preferably, the gas conduit comprises a gas inlet port in fluid communication with a gas source. The gas source may be a gas tank, bottle or main connection to an external gas tank, for example an oxygen bottle. The gas inlet port may be formed on the gas conduit, for example integrally, or may be secured to the gas conduit. The gas inlet port may be formed between the distal portion and the proximal portion of the conduit. Alternatively, the gas inlet port may be formed with or secured to the mask.

According to a second aspect of the invention, there is provided a respiratory support device comprising a gas conduit as described herein. In a preferred embodiment, the respiratory support device is a non-rebreather mask.

According to a third aspect of the invention, there is provided a mask comprising a gas conduit as described herein.

According to a fourth aspect of the invention, there is provided a gas reservoir comprising a gas conduit as described herein.

According to a fifth aspect of the invention, there is provided a method for preventing obstruction of a fluid conduit and/or for increasing fluid passage between two components (e.g. of a medical device) comprising the step of fluidly connecting the two components with a conduit as described herein.

The invention will be further described with reference to the drawings and figures, in which

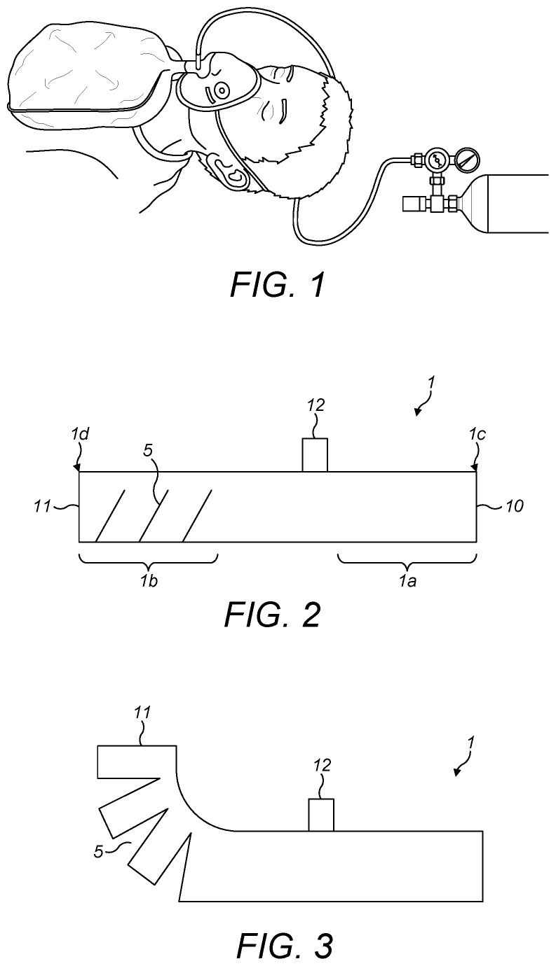

FIG. 1 is a schematic representation of an oxygen mask of the non-rebreather type;

FIG. 2 is a schematic representation of a conduit according to the present invention;

FIG. 3 is a schematic representation of another conduit according to the present invention;

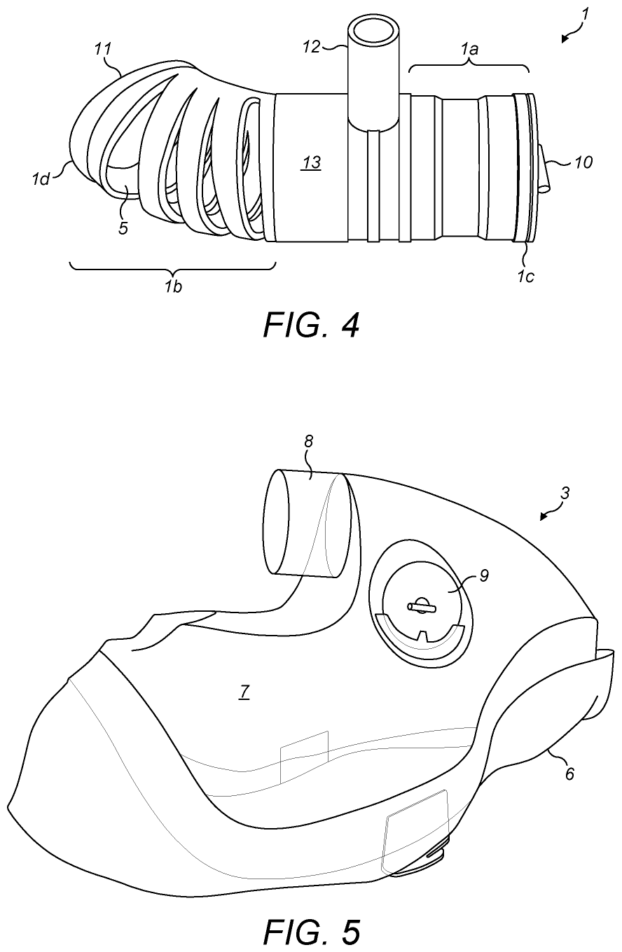

FIG. 4 is a schematic representation of another conduit according to the present invention;

FIG. 5 is a schematic representation of a face mask for use with the present invention;

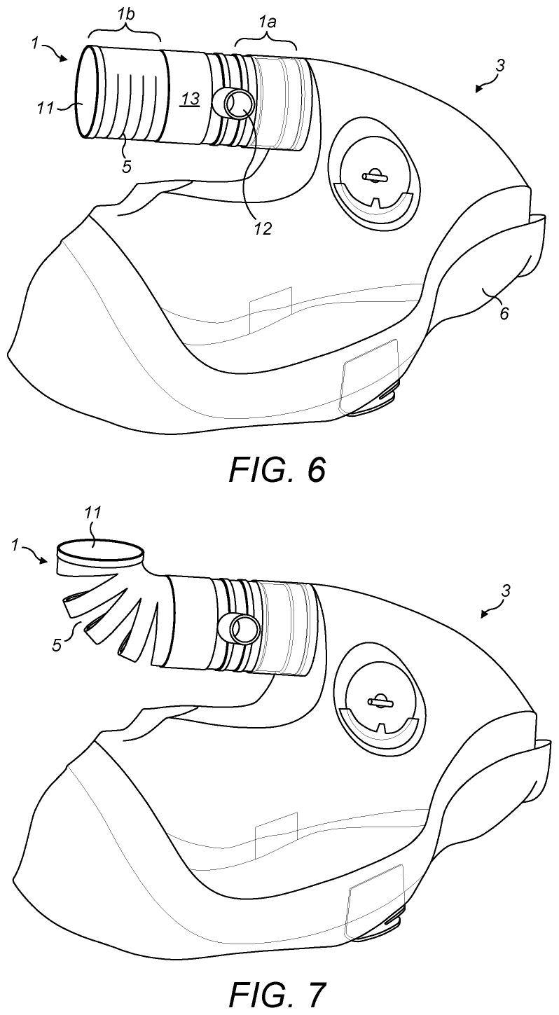

FIG. 6 is a schematic representation of the conduit of FIG. 4 (in a first configuration) connected to a face mask of FIG. 5;

FIG. 7 is a schematic representation of the conduit of FIG. 4 (in a second configuration) connected to a face mask of FIG. 5;

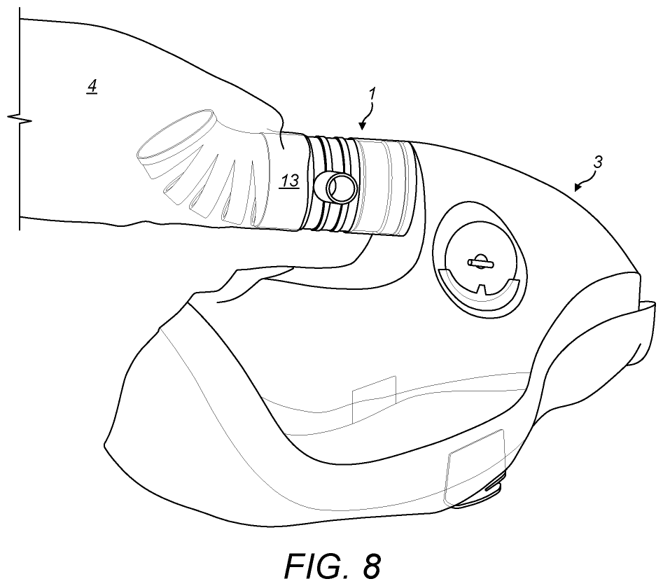

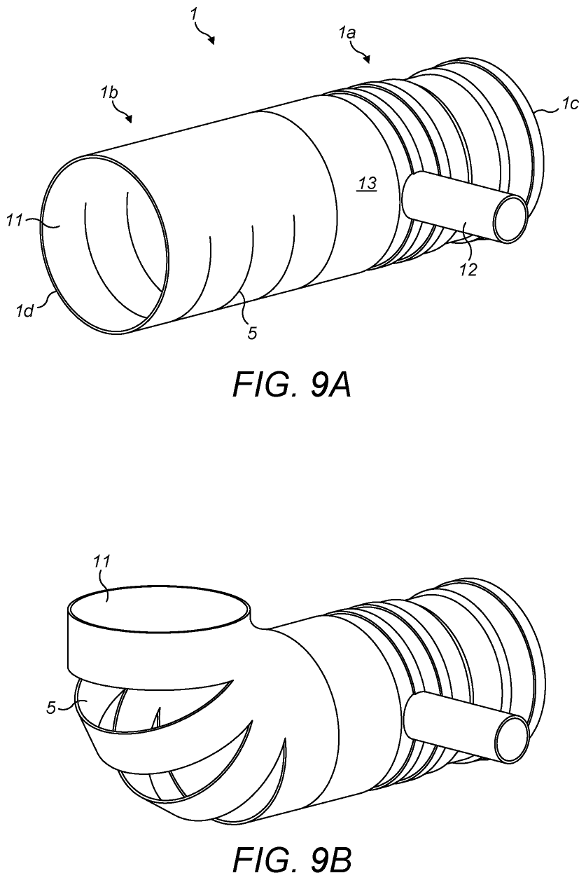

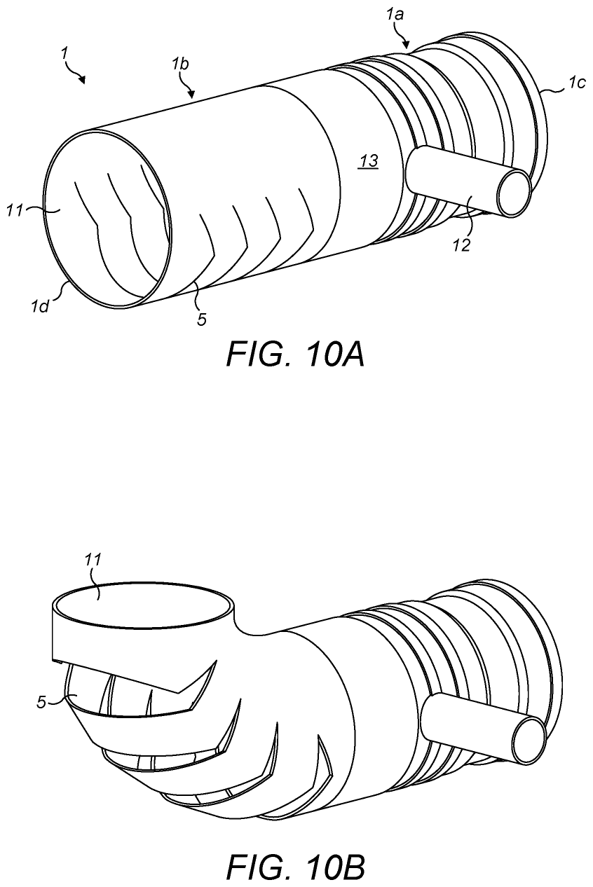

FIG. 8 is a schematic representation of a respiratory support device according to the present invention;

FIGS. 9A and 9B are schematic representations of a conduit according to the present invention in a first and second configuration, respectively;

FIGS. 10A and 10B are schematic representations of another conduit according to the present invention in a first and second configuration, respectively;

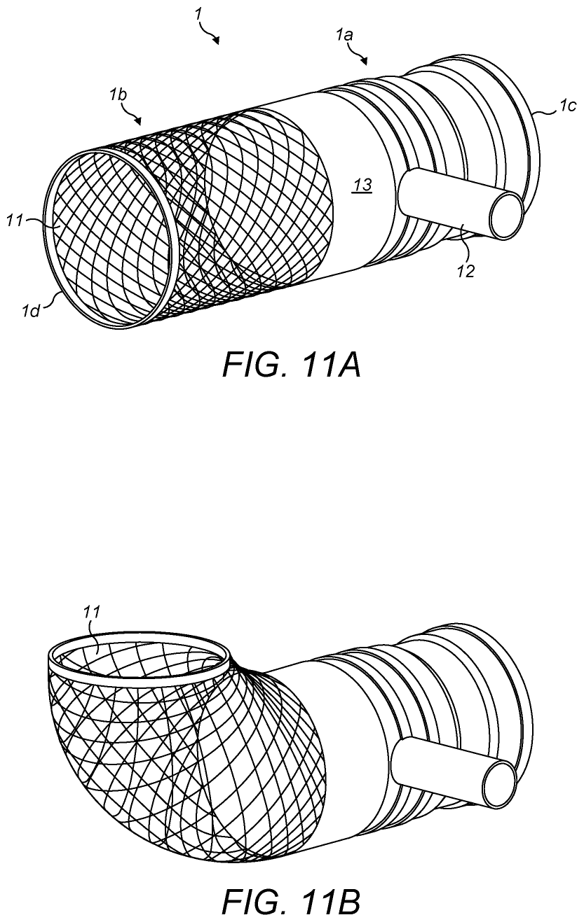

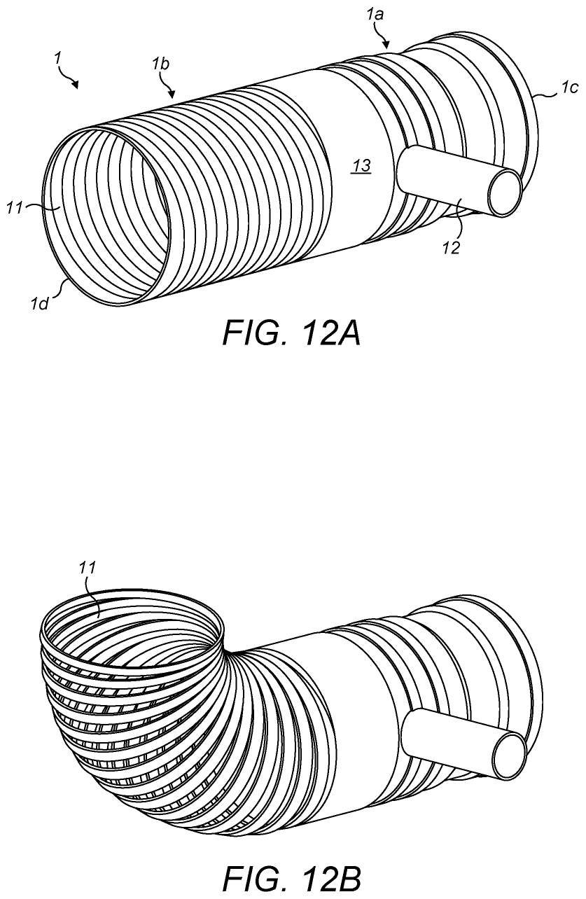

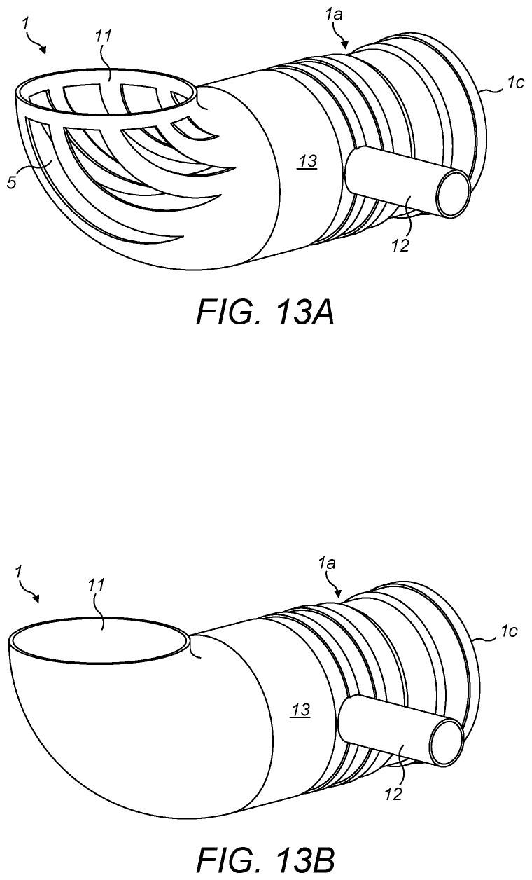

FIGS. 11A and 11B are schematic representations of another conduit according to the present invention in a first and second configuration, respectively;

FIGS. 12A and 12B are schematic representations of another conduit according to the present invention in a first and second configuration, respectively;

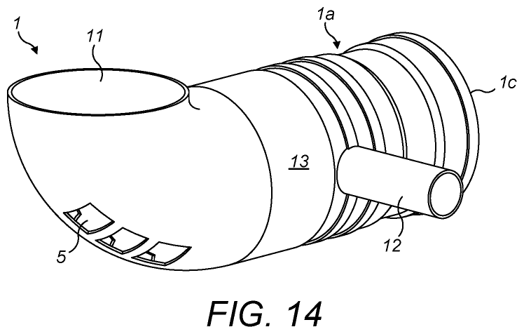

FIG. 13A is a schematic representation of another conduit according to the present invention;

FIG. 13B is a schematic representation of another conduit according to the present invention;

FIG. 14 is a schematic representation of another conduit according to the present invention; and

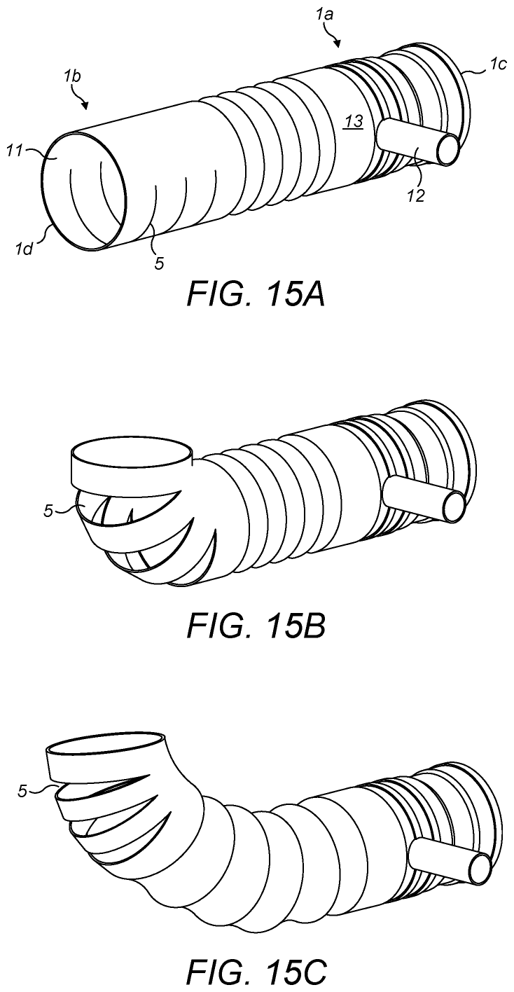

FIGS. 15A, 15B and 15C are schematic representations of another conduit according to the present invention in different configurations.

The exemplary devices described hereinafter are provided for the purpose of illustrating features of the present invention. The present invention relates, in particular to a conduit which may be connected to the mask (or to the neck or connector thereof) and extends into the reservoir bag. The present invention also relates to a respiratory support device comprising said conduit. The conduit of the present invention is described with reference to an exemplary respiratory support device.

A known respiratory support device is illustrated in FIG. 1, in the form of an oxygen mask of the non-rebreather or non-rebreathing type. The device comprises a mask applicable to the face of the patient, covering the mouth and nose of the patient. The mask comprises a neck or connector which enables gas communication with an oxygen reservoir bag. The neck comprises an oxygen line through which oxygen from an oxygen tank or bottle is delivered.

With reference to FIG. 8, there is illustrated a gas conduit 1 according to the present invention for a respiratory support device 2 comprising a face mask 3 and a gas reservoir bag 4, said gas conduit 1 comprising a proximal portion 1a for fluid connection to the mask and a distal portion 1b extending into the reservoir bag 4, wherein at least a side wall of said distal portion 1b comprises one or more apertures 5.

The mask 3, illustrated in FIGS. 5 to 8, is arranged and configured to cover the mouth and nose of the patient. The mask 3 comprises an opening 6 and an inner space 7 shaped and dimensioned so as to fit the contours of the face against and above which it is positioned.

The mask 3 may comprise a neck 8, usually positioned towards the uppermost area of the mask 3 above the nose of the patient. The neck 8 may serve or comprise means for securing with the conduit 1 and/or the reservoir 4. The neck 8 and/or connection means may be integrally formed with the mask 3. Alternatively, the neck 8 and/or securing means may be formed separately and connected to the mask 3.

Preferably, the mask 3 comprises one or more one-way valves 9, which enables the escape of the carbon dioxide exhaled by the patient from the inner space 7 of the mask 3 to the atmosphere, but prevents the patient from inhaling atmospheric air.

The mask 3 may comprise means for attaching the mask 3 to the patient's head, for example an elastic band secured to both sides of the mask and extending around the head of the patient, or more preferably an ear-loop elastic band attached on each side of the mask 3.

The reservoir 4, illustrated in FIG. 8, is preferably a reservoir bag, more preferably an oxygen reservoir bag. The reservoir bag 4 is preferably made of a plastics material. The reservoir 4 is in fluid communication with the mask 3, through an opening, which is hermetically sealed, for example to the mask 3. In practice, the health professional may apply adhesive tape to improve the seal. Preferably, the conduit 1 comprises a seal area 13 onto which the opening of the reservoir bag 4 may be sealed.

The conduit 1 preferably comprises an elongated member comprising an inner lumen for fluid flow therethrough. The cross-sectional outer shape of the conduit 1 is preferably circular, but may be any other suitable shapes including oval. Angle-free cross-sectional shapes are preferred. The elongated member may be formed integrally, or of separate components attached to each other.

In a preferred embodiment, the conduit 1 comprises a cylindrical member. Preferably, the conduit 1 is as thin as possible, whilst providing rigidity. Preferably, the side wall(s) of the conduit 1 is no more than 5 mm thick. Numerical ranges described herein include the lowest and highest values of the range.

The conduit 1 comprises a proximal portion 1a and a distal portion 1b.

The proximal portion 1a is closer to the mask 3, and, in this embodiment, is secured to the neck 8 of the mask 3. The conduit 1 may comprise means for securing the conduit 1 to the mask 3. Examples of securing means include, but are not limited to, screwing means, snap-fit, tab(s) and corresponding recess(es) and other mechanical securing means. The proximal portion 1a of the conduit may be made of a flexible material, and have outer dimensions slightly smaller than the inner dimensions of the receiving portion 8 of the mask 3, so that the proximal portion 1a can be pushed into and secured to the receiving portion 8. The conduit 1 may be welded, glued, adhered to the mask 3. Alternatively, the conduit 1 may be integrally formed with the mask 3. In a preferred embodiment, the proximal portion 1a of the conduit comprises one or more ribs, which can be received into one or more corresponding recesses in the (neck 8 of the) mask 3, in a snap-fit manner.

The conduit 1 preferably comprises a one-way valve 10. More preferably, the valve 10 is positioned in the proximal portion 1a, and most preferably at the proximal end of the conduit 1 (i.e. at the proximal opening 1c of the conduit 1). The one-way valve 10 enables oxygen to flow from the reservoir 4 to the mask 3 to be inhaled by the patient, but prevents exhaled carbon dioxide exhaled by the patient from flowing from the mask 3 to the reservoir 4.

The distal portion 1b is further from the mask 3, and extends beyond the neck 8 of the mask 3, into the reservoir bag 4. Therefore, the length of the conduit 1 should preferably be sufficient to extend beyond the opening of the mask 3 towards the bag. Owing to the presence of apertures in the distal portion 1b of the conduit 1, the maximum length of the conduit 1 is only limited by the length of the reservoir bag 4. Preferably, the proximal portion 1a inside the neck 8 has substantially the same length as the length of the neck 8 of the mask 3.

The distal portion 1b of the conduit 1 comprises one or more apertures 5, on the side wall of the conduit 1. In other words, the aperture 5 is not located at the distal end 1d of the conduit 1. In known respiratory support devices, gas flows through the single opening at the distal end of a rigid cylinder, or directly through the neck of the mask. These distal end openings are frequently partially or completely obstructed either by the reservoir bag, by the patient's anatomy, by covers and clothing and/or other obstacles, so that the flow of gas is impeded. A conduit 1 having one or more apertures 5 through the side wall(s) provides alternative and additional gas flow paths, thereby decreasing the risk of flow hindrance. Moreover, the apertures 5 increase the area through which the gas can flow, thereby enabling a greater flow capacity.

The shape of the apertures 1 is not limited. The apertures 5 may have the same or different shapes (FIGS. 13A and 14). The apertures 5 may be discrete apertures, or may be connected to other apertures. The apertures 5 may be formed as apertures through the side wall of the conduit 1, or may be apertures through a mesh (FIGS. 11a and 11b), through rings or spirals (FIGS. 12a and 12b), and the like.

In a preferred embodiment, the apertures 5 are through-slits, as shown for example in FIGS. 9a, 9b, 10a and 10b. The through-slits may comprise straight lines, undulations, zig-zags, jagged lines, crenellations, and the like.

Preferably, the through-slits 5 are formed along a partial (i.e. less than 100% of the) cross-sectional perimeter of the conduit 1. More preferably, the through-slits 5 are formed along more than 50% of the cross-sectional perimeter of the conduit 1.

The through-slits 5 may be formed perpendicularly relative to the longitudinal axis of the conduit 1 (as seen in FIGS. 3, 4 and 9), or they may be formed at an angle of less than 180 degrees, preferably 45 degrees (as seen in FIG. 2), relative to the longitudinal axis of the conduit 1. The through-slits 5 are preferably substantially parallel to each other so as to facilitate bending and shaping the distal portion 1b of the conduit 1.

When the respiratory support device 2 is fitted onto the patient, the conduit 1 comprises a frontal portion, facing away from the patient, a dorsal portion, facing towards the patient, and side portions between the frontal and dorsal portions. The one or more apertures 5 may be positioned in one or more of the frontal, dorsal and side portions of the conduit 1.

Frontal and side apertures 5 are more advantageous in that the obstructions are more commonly located on the patient's side of the device 2. Dorsal apertures 5 are more advantageous in that they may facilitate the bending and shapability of the conduit 1 away from the patient.

Preferably, the distal portion 1b of the conduit 1 is flexible and/or bendable. Preferably, the distal portion 1b of the conduit 1 may take a first configuration, in which the distal portion 1b is substantially straight, and a second configuration different from the first configuration. More preferably, the distal portion 1b of the conduit 1 may take a first configuration, in which the distal portion 1b is substantially straight and the apertures 5 are closed, and a second configuration different from the first configuration, in which the apertures 6 are opened. Preferably, the distal portion 1b of the conduit 1 is shapable, preferably manually shapable, in that it can retain a given configuration.

In FIGS. 9a and 9b, the apertures 5 are through-slits formed on the dorsal side of the conduit 1. FIG. 9a shows the distal portion 1b in a first configuration, in which the distal portion 1b is substantially straight and the through-slits 5 are closed. FIG. 9b shows a second configuration, in which the distal portion 1b is bent away from its first configuration and the through-slits 6 are opened. FIGS. 10a and 10b show two configurations of a conduit 1 comprising jagged through-slits 5. In FIGS. 11a and 11b, the distal portion 1b comprises or consists of a flexible and/or shapable mesh. In FIGS. 12a and 12b, the distal portion 1b comprises or consists of a spiral. Alternatively, the distal portion 1b of the conduit 1 may comprise a plurality of ribs and/or rings linked to each other by a bendable and/or shapable spine. FIGS. 13 and 14 show conduits 1 comprising a distal portion 1b, with a “fixed” shape, i.e. which is not shapable.

The distal portion 1b of the conduit 1 may be flexible enough so as to be bendable without impeding gas flow within the lumen of the conduit 1. Preferably, the conduit 1, of at least the distal portion 1b thereof, is made of a flexible material. Preferred materials include plastics materials.

The conduit 1 (or the distal portion 1b thereof) may comprise one or more pre-formed bends, or preferably it is bendable. Bendability may be achieved by various means, including, but not limited to, using one or more portions of flexible material (i.e. selecting a suitable flexible material), one or more hinges (i.e. mechanical means to bend the conduit 1), spirals, ribs and/or rings, a bendable spine, an accordion-type portion (as shown in FIGS. 15a, 15b, 15c) and the like.

The distal opening 11 may be formed at the distal end 1d of the conduit 1. Alternatively, it may be formed in a side wall of the distal portion 1b of the conduit 1, preferably on the frontal side of the conduit 1 (as illustrated in FIGS. 13 and 14). The distal opening 11 is preferably aligned with the side wall of the conduit 1, but may alternatively elevated relative to the side wall of the conduit 1.

In some embodiments, the conduit 1 does not comprise a distal opening at the distal end 1d of the conduit 1. In such embodiments, the distal end 1d of the conduit 1 is preferably rounded or curved so as to facilitate movement and/or bending of the distal portion 1b of the conduit 1 when it encounters an obstacle.

In the embodiments shown in FIGS. 13A, 13B and 14, the conduit 1 is provided with a single configuration, i.e. it is not bendable or shapable. The opening 11 is provided on the frontal side of the conduit 1. The opening 11 may be aligned with or made through the side wall of the conduit 1, or it may be offset from the side wall of the conduit 1 as shown in FIGS. 13A and 14.

In preferred embodiments, the distal opening 11 is formed and positioned at the distal end 1d of the conduit 1 in a first configuration, and positioned on the frontal side of the distal portion 1b in a second configuration.

The conduit 1 may comprise a gas inlet port 12, for delivering gas from the gas source to the reservoir 4. The gas inlet port 12 is positioned on the proximal portion 1a of the conduit 1, i.e. outside the reservoir bag 4. The gas inlet port 12 may be integrally formed with the conduit 1. In another embodiment, the conduit 1 does not comprise a gas inlet port and, instead, the gas inlet port is connected to or integrally formed with the mask 3.

In an exemplary manufacturing method (for example with reference to FIGS. 4 to 8), the main body of the conduit 1 (excluding the valve 10) may be integrally moulded from suitable plastics material. The apertures 5 may be subsequently form by cutting, perforating, punching or any other suitable method.

The valve 10 may be fitted, secured (e.g. by applying an adhesive, snap fit or other mechanical securing means) and sealed to the proximal end 1c of the conduit 1.

The proximal portion 1a of the conduit 1 is inserted into the neck 8 of a mask 3, until a rib of the conduit 1 fits into a corresponding recess formed in the inner surface of the neck 8 of the mask 3.

A tubing is secured to the gas inlet port 12. A gas reservoir bag 3 is provided separately, or sealably connected to the mask 3 or conduit 1.

In use, the mask 3 is positioned over the mouth and nose of the patient, and secured to the patient's head by means of elastic band(s). The tubing is connected to a gas source, e.g. an oxygen tank.

The health practitioner adjusts the flow of oxygen exiting the oxygen tank to that prescribed, and checks whether the mask 3 is fitted so that there is minimal leakage through potential gaps between the mask 3 and the face of the patient.

The distal opening 11 of the conduit 1 is checked for obstructions. If the distal opening 11 is obstructed, the health practitioner may manually bend the conduit 1 so that the distal opening 11 is positioned away from the obstruction.

The conduit 1 is bent from a first configuration (as shown in FIG. 6) to a second configuration (as shown in FIG. 7). In the first configuration, the distal opening 11 may obstructed by the patient's own anatomy, clothes or covers. In the second configuration, there is no hindrance of the distal opening 11, and gas can freely flow through the distal opening 11. The apertures 5 open as the conduit 1 is bent, and facilitate the bending of the conduit 1. Moreover, increased oxygen delivery is achieved through the combination of the distal opening 11 and the apertures 5.

During use, the partially inflated reservoir bag 4 may potentially fold over the distal opening 11, thereby reducing gas flow through the distal opening 11. However, gas will continue to flow through the apertures 5, so that oxygen delivery to the patient is never interrupted.

Thus, from the above description, it can be seen that the conduit of the present invention provides an improved gas delivery from a gas source to the reservoir, and from the reservoir to the mask. The present invention provides a simple solution to the problem of diminished flow due to the partial or complete obstruction of a conduit opening by obstacles (such as the patient's anatomy, clothing or cover). Consequently, the gas is delivered to the patient's airways in an efficient manner, with minimal loss and leakage, as prescribed by the health professional.

Although the present invention has been described in the context of respiratory support devices, in particular a non-rebreather oxygen mask, it is envisaged that it could have other advantageous implementations in other devices and systems, medical (e.g. inhalers) or not, requiring an improved flow of fluid as described herein.

Similarly, although the present invention has been described in relation to a gas conduit, the conduit may be a fluid conduit, wherein the conduit is a gas, liquid or a mixture thereof.

The following description describes a ribbed cylinder, as described in the priority application, and forms part of the present disclosure.

### FIELD OF APPLICATION

The invention is a connecting component between a mask and an oxygen reservoir.

I refer here to an extract from the chapter on oxygen treatment in the Emergency Medicine Handbook [Legevakthåndboken]: “In acute medical treatment away from hospital, oxygen is an important drug. Ample oxygen supply is often recommended. The treatment aim should be O2 saturation >90-95, depending on condition. Choose a method of administration that is suitable for the amount of oxygen the patient needs. The following alternatives are available:

Oxygen by nasal catheter/nasal cannula, give 2-4 l/min. Gives approx. 30% oxygen.

Oxygen by mask without reservoir, give 6-8 l/min. Gives approx. 50% oxygen.

Oxygen by mask with reservoir, give 8-12 l/min (until the reservoir is full). Gives approx. 70% oxygen. Often preferred in awake critically ill or injured patients who are breathing for themselves.

Indication: hypoxia, respiratory failure, circulatory failure, severe trauma.”

### STATE OF THE ART—TECHNIQUES ON WHICH THE INVENTION IS BASED

The existing connecting tube between the mask and the reservoir bag is a rigid plastic cylinder. The point for connecting the oxygen hose from the oxygen bottle is perpendicular, centrally, to the cylinder. In some variants a joint is made in the fixing point to the mask, so that the cylinder with the bag can be angled in relation to the mask. This solution does not prevent the risk of pinching of the neck of the reservoir bag. The same applies to variants where the rigid plastic cylinder is replaced with a flexible accordion tube. The accordion solution does not actually reduce the risk of pinching in the neck of the bag. Nor does the accordion solution increase the area for oxygen flow into and out of the bag. The accordion solution has soft walls, which in itself presents a risk of pinching.

Examples of Other Methods for Bending Rigid Material

To create curves in a wooden plank for the wing board on a mansard roof, the builder makes transverse, but not through, cuts on one side of the wooden board, several one after another, so that the rigidity of the wooden board is weakened. The wooden board can then be curved more easily.

Alternatively, the piece of wood must be heated in a steam chamber and bent in a clamp, so that the wooden board remains bent when it has dried, as in boat building.

A suction tube with accordion segment means that the suction tube can be used without difficulty regardless of the angle that the end openings make with one another.

A “slinky” staircase toy at rest is in the form of a solid cylinder, but the walls are a spiral, which can expand and open when the upper turns are raised.

Our thorax is in the form of a solid cylinder, but is built up of movable ribs with fixed points in the thoracic spinal column.

### IMPROVEMENT OF THE PRIOR ART

Oxygen treatment during patient transport is decisive for survival and the risk of sequelae after illness or injury. During ambulance transport with oxygen treatment there is a known risk of pinching in the neck of the oxygen reservoir bag. The cause is as a rule the patient's bed during transport, plus clothing and bedclothes, which are located higher than the mask cylinder. Pinching in the neck of the bag has the result that potential oxygen supply to the patient is more than halved. The invention is characterized in that it is a flexible connecting cylinder between the mask and the oxygen reservoir bag, which reduces the risk of pinching of the neck of the reservoir bag, and at the same time multiplies the area for flow of gas in and out of the reservoir bag. The invention is characterized in that it is possible to use the same reservoir bag and the same mask as before, but they are connected together by means of the invention.

### NECESSARY MEANS FOR PRODUCTION

The invention is characterized in that the currently existing rigid cylinder can be worked. The invention is characterized in that it can be used together with, and be connected to, the currently existing reservoir bag and mask. The invention is characterized in that one or more cuts, e.g. slanting or perpendicular, are made in the end of the cylinder that goes into the reservoir bag. The invention is characterized in that the cuts are not through-cuts, and that the upper portion of the cylinder wall is intact, so that the cylinder wall maintains its integrity. The invention is characterized in that at the same time as the end piece is bent, the cylinder walls open on the inside of the reservoir bag. The invention is characterized in that the cuts in the cylinder wall open when the cylinder is bent. The invention is characterized by a manual increase in the area of the opening in the reservoir bag, and at the same time the invention creates a framework that strengthens the neck of the bag. The invention is characterized in that the increased opening into the reservoir bag gives increased flow in and out of the reservoir bag, and the invention reduces the risk both for and in pinching of the neck of the bag. The invention is characterized in that the reservoir bag is pulled over the end of the cylinder, which has one or more rib-like cuts. The invention is characterized in that the whole segment with cuts on the cylinder is covered and sealed inside the reservoir bag, in such a way that leakage from the connection between the reservoir bag and the cylinder is negligible.

### EMBODIMENT EXAMPLES—ILLUSTRATION

FIG. 1 shows an illustration from the Emergency Medicine Handbook [“Legevakthåndboken”]: Mask with oxygen reservoir. The green hose from the oxygen tank is fastened to the connecting cylinder, which is the connecting element between the mask and the reservoir bag.

The invention is illustrated in side view in FIG. 2 and FIG. 3, in which illustrations 3 slanting cuts have been made in the end piece.

FIG. 2 shows the invention, ribbed cylinder, with 3 rib-like cuts, in the unbent and closed position.

FIG. 3 shows the invention, ribbed cylinder, which is bent so that the rib-like cuts open.

### SUMMARY

Ribbed cylinder is a cylinder with transverse cuts applied in one end piece (FIG. 2). The end piece with ribbing can be introduced into the neck of the reservoir bag and fastened to the reservoir bag by existing methods. During oxygen treatment with a mask and a reservoir (FIG. 1), the ribbing in the end piece will be bent so that the ribbing opens, and the area for flow of gas in and out of the reservoir bag is multiplied (FIG. 3). The curvature of bending of the end piece (FIG. 3) moves the reservoir bag away from potential pinching, contributes to support and structure in the neck of the reservoir bag, and reduces the risk for and in pinching over the end of the cylinder.

The following numbered statements set out particular combinations of features which are considered relevant to particular embodiments of the present disclosure:
1. Ribbed cylinder, characterized by one or more cuts or perforations at the end of a cylinder in such a way that an object attached to the end piece will move in the same direction.
2. Ribbed cylinder according to Statement 1, used as coupling between the mask and the reservoir bag for oxygen treatment, characterized in that one or more cuts or perforations are made in the end of the cylinder that is introduced into the reservoir bag, so that the cylinder can be bent inside the reservoir bag.
3. Ribbed cylinder according to Statements 1-2, characterized in that the reservoir bag can be angled up to 90 degrees in relation to the mask when the ribbing in the cylinder opens.
4. Ribbed cylinder according to Statements 1-3, characterized in that the cylinder wall in the region with ribbing gives increased area for flow of gas when the cylinder wall is bent and the ribbing opens.
5. Ribbed cylinder according to Statements 1-4, characterized by a rigid cylinder that maintains its angled position with open ribbing.
6. Ribbed cylinder according to Statements 1-5, characterized in that it can also be bent manually when the reservoir bag is attached.
7. Ribbed cylinder according to Statements 1-6, characterized in that in the open position it gives support and integrity to the neck of the reservoir bag, so that the neck of the reservoir bag maintains its opening and ensures flow of gas between the reservoir and the mask.
8. Ribbed cylinder according to Statements 1-7, characterized in that it reduces the risk of pinching in a case where the reservoir bag is located over the end of the cylinder, because the bent and open cylinder has a greatly increased area for flow of gas in the reservoir bag.
9. Ribbed cylinder according to Statements 1-8, characterized in that the reservoir bag can be angled in relation to the mask, so that the reservoir bag is turned away from potential pinching obstructions during oxygen treatment.
10. Ribbed cylinder according to Statements 1-9, characterized in that for implementing the invention it is possible to use equivalent materials and dimensions as for an existing component, and make the rib-like cuts/perforations in the end of the cylinder that is to be in the neck of the reservoir bag.

1. A gas conduit for a respiratory support device comprising a face mask and a gas reservoir bag, said gas conduit comprising a proximal portion for fluid connection to the mask and a distal portion extending into the reservoir bag, wherein at least a side wall of said distal portion comprises one or more apertures.

2. The gas conduit according to claim 1, wherein the distal portion of the gas conduit is made of a flexible material.

3. The gas conduit according to claim 1 or 2, wherein the gas conduit comprises or consists of a shapable material or structure.

4. The gas conduit according to any one the preceding claims, wherein the gas conduit comprises a frontal side and a dorsal side, and the apertures are formed at least on the dorsal side.

5. The gas conduit according to any one the preceding claims, wherein the apertures are through-slits.

6. The gas conduit according to any one the preceding claims, wherein the distal portion of the gas conduit comprises a plurality of ribs.

7. The gas conduit according to any one the preceding claims, wherein the distal portion of the gas conduit comprises a plurality of rings.

8. The gas conduit according to any one the preceding claims, the distal portion of the gas conduit comprises a mesh.

9. The gas conduit according to any one the preceding claims, the gas conduit comprises a distal opening formed on the frontal side of the conduit.

10. The gas conduit according to any one the preceding claims, wherein the gas conduit comprises a rounded distal end.

11. The gas conduit according to any one the preceding claims, wherein the distal and proximal portions of the gas conduit are integrally formed.

12. The gas conduit according to any one the preceding claims, wherein the gas conduit comprises means for securing the proximal portion of the gas conduit to an element of the mask.

13. The gas conduit according to any one the preceding claims, wherein the gas conduit comprises a valve to regulate the gas flow between the mask and the reservoir bag.

14. The gas conduit according to any one the preceding claims, wherein the gas conduit comprises a gas inlet port in fluid communication with a gas source.

15. A respiratory support device comprising a gas conduit as defined in any one of the preceding claims.

16. The respiratory support device according to claim 15, wherein the respiratory support device is a non-rebreather mask.
